(19) United States
(12) Patent Application Publication
(10) Pub. No.: US 2013/0281808 A1
(43) Pub. Date: Oct. 24, 2013

(54) BLOOD COMPONENT DETECTION DEVICE

(71) Applicant: Dar-Bin SHIEH, Tainan City (TW)

(72) Inventor: Dar-Bin SHIEH, Tainan City (TW)

(73) Assignee: National Cheng Kung University, Tainan City (TW)

(21) Appl. No.: 13/451,683

(22) Filed: Apr. 20, 2012

(51) Int. Cl. A61B 5/00

(52) U.S. Cl. 600/365

(57) ABSTRACT

The present invention relates to a blood component detection unit, which comprises: a capsule body having a first surface; a needle array including a plurality of needles, each of the needles have an opening and an inner hollow space, and each of the needles protrudes out of the first surface; a detection chamber disposed inside the capsule body and connected with the inner hollow space; a sensing chip disposed inside the capsule body; and a plurality of sensing elements disposed on the sensing chip.

### BACKGROUND OF THE INVENTION

1. Field of the Invention

The present invention relates to a blood component detection unit and a blood component sampling equipment, especially an easy-to-operate and portable blood component detection unit.

2. Description of Related Art

Recently, the number of patients suffering from chronic disease rises continuously. Take diabetes for example, people with diabetes need to check their health condition regularly (for example: blood detection of blood sample from the patients once in every 8 hours). The blood detection refers to the concentration of a specific chemical composition in the blood of the patients. To the diabetic patients described above, the blood glucose concentration needs to be examined. Therefore, for diabetic patients, the blood glucose concentration needs to be examined every 8 hours.

Since the detection of blood glucose concentration is extremely important to diabetic patients, and it is necessary to be executed every 8 hours everyday, the execution or the steps should be as simple as possible so that each of diabetic patients can detect and track their own health condition easily and simply. More importantly, the methods known in the art of obtaining the blood sample is through drawing blood, which increases the pain of patients and causes the patients to purposefully avoid the examination of blood glucose concentration.

In addition, in order to maintain the accuracy of the blood glucose concentration detection, certain amount of blood is usually be drawn during the process of blood drawing, therefore, the lancet is usually used in the process of blood drawing to quickly collect certain amount of blood. However, the aggravation of the pain of patients further decreases the patients' will to execute the detection of blood glucose concentration. Especially for elderly patients, the exclusion of detecting blood glucose concentration of elderly patients will be affected because of the pain.

As a result, detecting and monitoring blood glucose concentration of diabetic patients will not be taken effectively, and the health condition of diabetic patients could not be effectively controlled.

Therefore, for diabetic patients, it is great news to develop an easy-to-operate, and portable blood component detection unit, which improve the pain caused by obtaining the blood sample.

### SUMMARY OF THE INVENTION

The object of the present invention is to provide a blood component detection unit, which is portable, easy-to-operate, and easy to further improve the pain control during blood sampling.

In order to achieve the purpose described above, the present invention provides a blood component detection unit, comprising: a capsule body having a first surface; a needle array including a plurality of needles, each of the needles having an opening and an inner hollow space, and each of the needles protrudes out of the first surface; a detection chamber disposed inside the capsule body and connecting with the inner hollow space; a sensing chip disposed inside the capsule body; and a plurality of sensing elements disposed on the sensing chip.

The shape of the above described blood component detection unit is not limited herein, however, in the blood component detection unit of the present invention, the tablet shape of the blood component detection unit is preferable. Further, the blood component detection unit is preferred to be disposable.

In addition, the material of the above-described needles is not particularly limited to a particular material type; however, the needles made of biocompatible materials for the blood component detection unit of the present invention is preferable. Furthermore, the material of the above-described capsule body is not limited, however, the capsule body made of biodegradable materials is preferable.

In addition, the needles coated with anesthetic are preferable, so that the patients will not feel pain when the needles pierce through the skin of patients because of the coating of anesthetic on the needles. It is noteworthy that the needles described above are prefer to be manufactured by imprinting process, LIGA process, and electroplating process or a combination of above and another noteworthy thing is that the mold used in the above-described imprinting process is preferably manufactured by Lithographie Galvanoformung Abformung Electroforming Micro Molding (LIGA) process.

The present invention also provides a blood component detection device, comprising: a strip having an upper surface; and a plurality of blood component detection units as described above, wherein the plurality of blood component detection units disposed on the upper surface. Wherein the plurality of blood component detection units is pasted on the first surface, and a distance between two adjacent blood component detection units is the same.

The present invention further provides a blood component sampling equipment, comprising: a housing having an opening; a blood component detection equipment as described above, which is disposed in the housing; an elastic element, which is disposed in the housing; and at least one roller element transporting the blood component detection unit.

The form of the elastic element is not limited thereby; any elastic elements are useable for the blood component sampling equipment of the present invention. However, the blood component sampling equipment of the present invention, wherein the elastic element is preferred to be a spring. In addition, the blood component sampling equipment further comprises a cap covering one end of the opening of the housing.

The present invention also provides another blood component sampling equipment, comprising: a housing; a cap disposed on the housing; a turntable component, which is disposed inside the housing and has a central hole, wherein the turntable component rotates around the central hole as the axis; a depth controlling component having a plurality of sub-sections, wherein each of the sub-sections has different height; a plurality of blood component detention assemblies, each of the blood component detection assemblies comprises a needle section and a supporting section; and a driving unit comprising a fixed part and a movable part, wherein fixed part has a pushing part; wherein the needle part is disposed on the pushing part.

The present invention further provides another blood component sampling equipment, comprising: a housing; a cap disposed on the housing; a turntable component disposed inside the housing and having a central hole, wherein the turntable component rotates around the central hole as the axis; a plurality of blood component detention assemblies, each of the blood component detection assemblies comprises a needle section and a supporting section; and a driving unit comprising a fixed part and a moveable part, wherein the fixed part is wound with a coil, and the moveable part is configured with a permanent magnet.

The blood component sampling equipment of the present invention, wherein the turntable component rotate in clockwise or counterclockwise direction.

### BRIEF DESCRIPTION OF THE DRAWINGS

FIG. 1A is a schematic diagram of the blood component detection unit of example 1 of the present invention.

FIG. 1B is a side view of the blood component detection unit of example 1 of the present invention.

FIG. 2 is a schematic diagram of the blood component detection device of example 2 of the present invention.

FIG. 3 is a schematic diagram of the blood component sampling equipment of example 3 of the present invention.

FIG. 4A is a side view of the blood component sampling equipment of example 4 of the present invention,

FIG. 4B is the first schematic diagram of the blood component sampling equipment of example 4 of the present invention.

FIG. 4C is the second schematic diagram of the blood component sampling equipment of example 4 of the present invention.

FIG. 4D is the first schematic diagram of the driving unit of the blood component sampling equipment of example 4 of the present invention.

FIG. 4E is the second schematic diagram of the driving unit of the blood component sampling equipment of example 4 of the present invention.

FIG. 4F is the third schematic diagram of the driving unit of the blood component sampling equipment of example 4 of the present invention.

FIG. 5A is the first schematic diagram of the driving unit of the blood component sampling equipment of example 5 of the present invention.

FIG. 5B is the second schematic diagram of the driving unit of the blood component sampling equipment of example 5 of the present invention. and

FIG. 5C is the third schematic diagram of the driving unit of the blood component sampling equipment of example 5 of the present invention.

### DETAILED DESCRIPTION OF THE PREFERRED EMBODIMENT

The exemplary embodiments of the present invention will be described in detail. For those of ordinary skill in the art, the advantages and effectiveness of the present invention can be easily realized through the contents disclosed in the present specification. In addition, the present invention can be embodied and practiced by the other different embodiments, and it is understood that many other possible modifications and variations can be made without departing from the spirit and scope of the invention. The following embodiments are described in order to enable those of ordinary skill in the art to embody and practice the present invention.

### Example 1

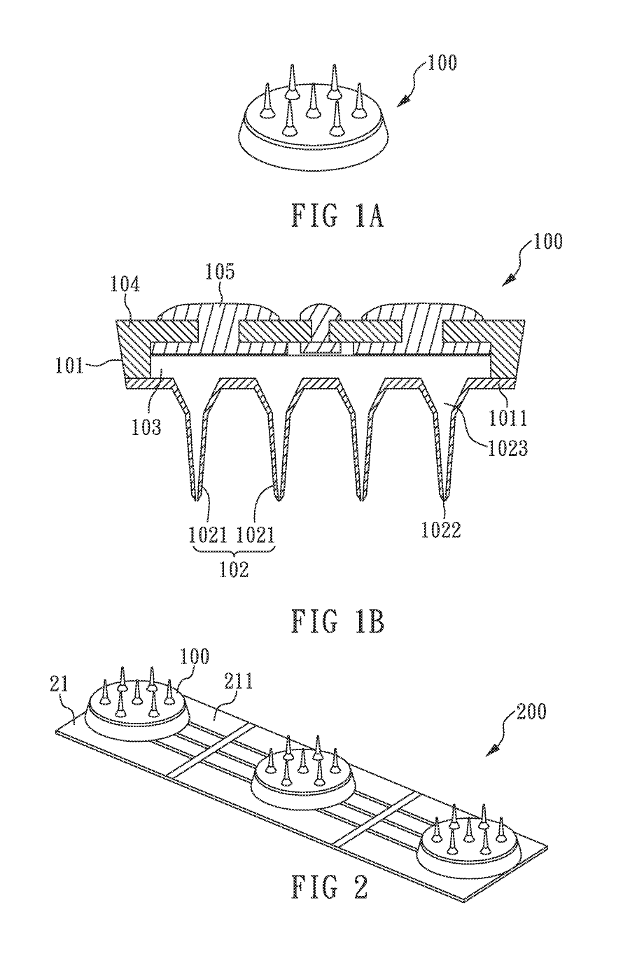

The blood component detection unit of example 1 of the present invention is shown in FIGS. 1A and 1B. FIG. 1A is a schematic diagram showing the blood component detection unit of example 1 of the present invention, and FIG. 1B is a side view of example 1 of the present invention.

As shown in FIGS. 1A and 1B, the blood component detection unit 100 of example 1 of the present invention comprises a capsule body 101, a needle array 102, a detection chamber 103, and a sensing chip 104.

The needle array 102 described above comprises a plurality of needles 1021. In addition, the capsule body 101 surrounds the detection chamber 103 and the sensing chip 104. Further, each needle 1021 protrudes from a first surface 1011 of the capsule body 101. As shown in FIG. 1A, the blood component detection unit 100 is in a tablet form.

As shown in FIG. 1B, the pinpoint of each of the needles 1021 has an opening 1022, which is used as an import opening for blood sample (such as tissue blood). In addition, each of the needles 1021 also has an inner hollow space 1023 used to receive the blood sample imported from the opening, and the inner hollow space 1023 is inside the needles. Furthermore, the inner hollow space 1023 inside each of the needles 1021 is connected with the detection chamber, so that the blood sample can be introduced to the detection chamber 103 through the inner hollow space 1023 while the sensing chip 104 described above can detect the blood sample. In addition, as shown in FIG. 1B, one or more inductor 105 is set on one surface of the sensing chip 104, and the inductor 105 described above faces the detection chamber 103. As described above, the number of the inductor 105 can be one or more, the blood component detection unit in the example 1 of the present invention comprises two inductors 105 to form an inductor 105 arrays.

The blood component detection unit 100 of example 1 of the present invention is disposable. Moreover, the needles 1021 of the blood component detection unit 100 is made of biocompatible materials, and the other parts of the blood component detection unit 100 is made of biodegradable materials. In addition to this, the above-described needles 1021 are manufactured by an imprinting process; wherein the mold used in the above-described imprinting process is manufactured by LIGA process in order to maintain the high accuracy of the size and the direction of each of the needles 1021 in the needle array 102. In addition, in order to improve the efficiency of collecting blood samples, the pinpoint of each of the needles 1021 has a special cone and ditch.

When the blood component detection unit 100 of the example 1 of the present invention is used to detect the existent and/or the concentration of the desired ingredients (for example, the specific chemical ingredients or the blood glucose concentration of the detected blood sample), the needle array 102 will be driven. The method to drive the needle array described above is not limited. It can be mechanically driven, electromagnetically driven, or manually driven methods. Further, the pinpoints of the needles 1021 are pushed to penetrate through a patient's skin. Each of the needles 1021 is coated with anesthetic, so that when the needles penetrate through the patient's skin, the patient will not feel pain.

Since the peripheral part of the needle array 102 is pressed into patient's skin earlier than the rest part of the needle array 102 does, the needles 1021 of the peripheral part of the needle array 102 penetrate deeper than the needles 1021 of the rest part of the needle array does and begin to collect the blood sample. Hereafter, the needles 1021 of the blood component detection unit 100 are detached from the patient immediately when the needles 1021 penetrate into patient's skin. At the same time, the space inside the detection chamber 103 forms a negative pressure, and further enables the blood component detection unit 100 to draw blood sample from the patient. The blood sample then passes through the opening 1022 of the needles 1021 and flows into the main body of the needles, and then is introduced into the detection chamber 103.

When the blood sample is introduced into the detection chamber 103, the blood sample flows into the inductor 105 and the sensing chip 104, the blood component detection unit 100 then executes the examination. Hereafter, the result of the examination from the sensing chip 104 is exported to a remote server to proceed signal processing and analysis. Wherein the result of the examination is the concentration of the blood glucose or the other specific chemical component of the blood sample described above. In addition, the remote server may be, for example, a microprocessor of a blood glucose meter.

### Example 2

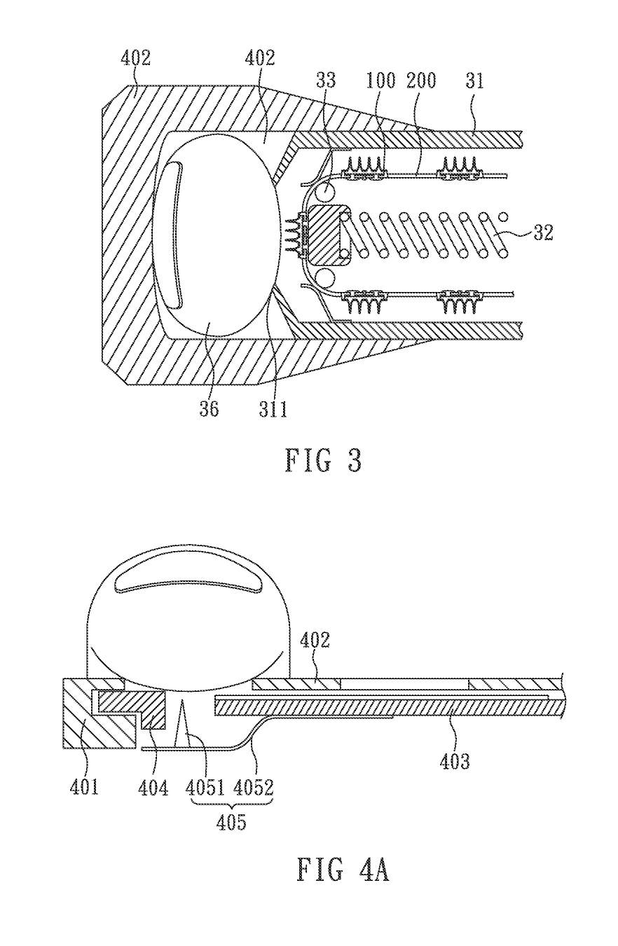

The blood component detection device of example 2 of the present invention is shown in FIG. 2. FIG. 2 is a schematic diagram showing the blood component detection device of example 2 of the present invention, the implementation of the blood component detection device of example 2 of the present invention is similar to the implementation of the blood component detection unit 100 of example 1 of the present invention. The difference is that the blood component detection device of example 2 of the present invention further comprises a strip 21 having an upper surface 211.

In addition, as shown in FIG. 2, a plurality of blood component detection unit 100 described in example 1 of the present invention is disposed on the upper surface 211. In addition, the blood component detection units are pasted on the upper surface, wherein the distance between two adjacent blood component detection units is identical. The other implementations, which are the same as example 1, will not be further described herein.

### Example 3

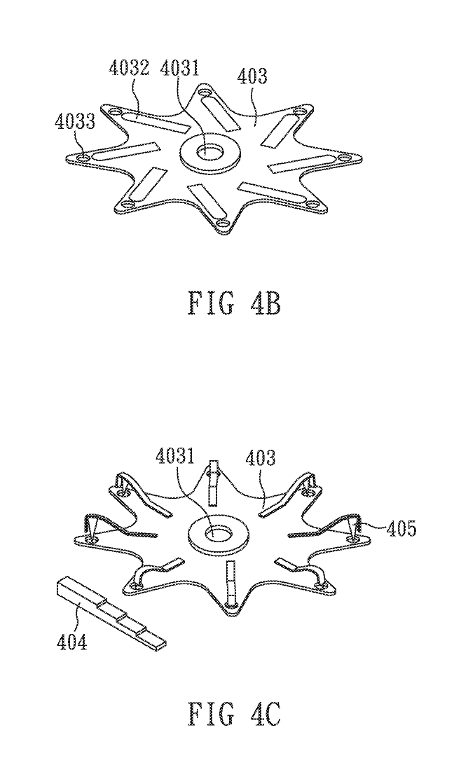

The blood component sampling equipment of example 3 of the present invention is shown in FIG. 3. FIG. 3 is a schematic diagram showing the blood component sampling equipment of example 3 of the present invention. As shown in FIG. 3, the blood component sampling equipment of example 3 of the present invention comprises: a housing 31, a blood component detection device 200 of the example 2 of the present invention, an elastic element 32, and at least one roller element 33.

The housing described above comprises an opening 311, and the blood component detection device 200 is disposed in the housing. Furthermore, the elastic element 32 is also disposed in the housing 31. As shown in FIG. 3, the roller element 33 is used to transport the blood component detection device 200, and one of the blood component detection units 100 of the blood component sampling device 200 protrudes out of the hosing 31 corresponding the opening 311.

As shown in FIG. 3, the blood component sampling equipment of example 3 of the present invention further comprises a cap 34, and the cap 34 preferably covers one end of the opening 311 of the housing 31 to form a capacity space 35. When the blood component sampling equipment of the present example is applied, the finger 36 of a patient is laid into or through the space 35.

Then, the elastic element 32 pushes the blood component detection unit 100 located at the opening 311 to penetrate the needles of the blood component detection unit 100 into the patient's skin and draw the blood sample. After the blood sample is collected, the elastic element 32 returns to the original location, and rotates the roller element 33. By the rotation of the roller element 33, the strip is driven to remove the used blood component detection unit 100 from the opening 311 and bring the unused blood component detection unit 100 to the opening 311 in order to proceed to the next examination.

Therefore, the used blood component detection unit 100 will be stored inside the housing, until all the blood component detection units 100 inside the blood component sampling equipment of example 3 of the present invention are used. The used blood component sampling equipment is then discarded. According to the good storage system described above, the biological wastes problem caused by improper disposal of the blood component detection units 100 will be improved.

### Example 4

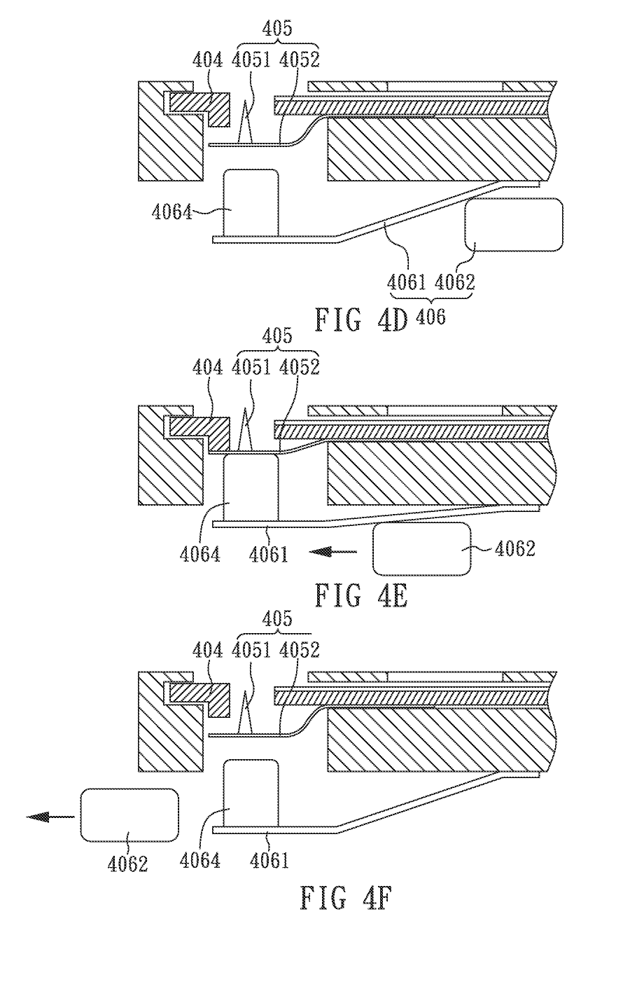

The blood component sampling equipment of example 4 of the present invention is shown in FIGS. 4A, 4B and 4C. FIG. 4A is a side view of the blood component sampling equipment of example 4 of the present invention, FIG. 4B is the first schematic diagram showing the blood component sampling equipment of example 4 of the present invention, and FIG. 4C is the second schematic diagram showing the blood component sampling equipment of example 4 of the present invention. As shown in FIG. 4A, FIG. 4B, and FIG. 4C, the blood component sampling equipment 400 of example 4 of the present invention comprises: a housing 401, a lid 402, a turntable component 403, a depth controlling component 404, a plurality of blood component detection assemblies 405, and a driving unit 406.

There is a central hole 4031 in the turntable component 403, and the central hole 4031 of the turntable component 403 matches the corresponding center of the housing 401. The turntable component rotates around the central hole as the axis. In addition, a plurality of openings 4032 is arranged on the turntable component 403. For the blood component sampling equipment of example 4 of the present invention, the number of the openings 4032 is 8. Furthermore, each opening 4032 has a window 4033.

In addition, each of the blood component detection assemblies 405 is arranged to correspond to an opening 4032 of the turntable component 403. The blood component detection assembly 405 has a needle section 4051, and a supporting section 4052.

Please refer to FIG. 4A. The lid 402 is located at the top of the housing 401. In addition, the depth-controlling component 404 described above is disposed inside the housing 401 in order to control the moving depth of the blood component detection assembly 405 toward the lid 402. In addition, as shown in FIG. 4C, the depth-controlling component 404 having a plurality of sub-sections, and the height of each of the sub-sections is different in order to control the different depth of the blood component detection assembly 405. That is, by moving the moveable depth-controlling component 404, the blood component detection assembly 405 will have a deeper moving depth. The method of driving the depth-controlling component is not limited thereby. For example, the movement of the depth-controlling component can be electrically driven or mechanically driven.

After the blood sample is collected, the turntable component 403 rotates to the position under the lid 402 of one of the plurality of opening 4032. Hereafter, the depth-controlling component is set to a predetermined position to control the moving depth of the blood component assembly 405.

Next, the related mechanism to move the blood component detection assembly 405 upward by the driving unit 406 will be described in detail. Please refer to FIGS. 4D, 4E, and 4F. FIG. 4D is the first schematic diagram showing the driving unit of the blood component sampling equipment of example 4 of the present invention, FIG. 4E is the second schematic diagram showing the driving unit of the blood component sampling equipment of example 4 of the present invention, and FIG. 4F is the third schematic diagram showing the driving unit of the blood component sampling equipment of example 4 of the present invention.

As shown in FIG. 4D, the driving unit 406 comprises a fixed part 4061 and a moveable part 4062, wherein the fixed part 4061 has a pushing part 4064. Further, as shown in FIG. 4D, the pushing part 4064 is disposed under the blood component detection assembly 405, and the needle part 4051 is disposed on the pushing part.

At first, as shown in FIG. 4D, the moveable part 4062 is at an original position as shown in the left diagram of FIG. 4D. Then, the moveable part 4062 moves to the left near the fixed part 4061, as shown in FIG. 4E, when the moveable part 4062 closes the fixed part 4061 gradually, the fixed part 4061 is lifted as well as the pushing part 4064, and the needle part 4051 and the supporting part 4052 of blood component detection assembly 405 are lifted together.

Please refer to FIG. 4E again. The needle part 4051 and the supporting part 4052 are not lifted unrestrictly. As shown in FIG. 4E, when the needle part 4051 and the supporting part 4052 are lifted to a particular height, the depth controlling component 404 will lock the supporting part 4052 to avoid the needle part 4051 and the supporting part 4052 be lifted unrestrictly.

As shown in FIG. 4F, the moving part 4062 moves to the left continuously until leaving the fixed part 4061. In this way, the fixed part 4061 is back to the position as shown in FIG. 4D. As described above, when the fixed part 4061 is lifted to the position shown in FIG. 4E, the needle part 4051 penetrates into a patient's skin to collect the blood sample. After the blood sample is collected, the turntable component 403 rotates to the next position where the next opening 4032 is under the lid 402. In addition, the method of rotating the turntable component 403 may be driven mechanically, electromagnetically, or manually. The rotation direction may be clockwise or counterclockwise.

When all the needle part 4051 of the blood component detection assembly 405 is used, the turntable component 403 can be removed from the housing 401, and be replaced by a unused turntable component 403.

### Example 5

The implementation of the blood component sampling equipment of example 5 of the present invention is similar to the implementation of the blood component sampling equipment of example 4 of the present invention, therefore, the following description, which is the focus on the different implementation between example 4 and example 5, the same implementation will not be reiterated.

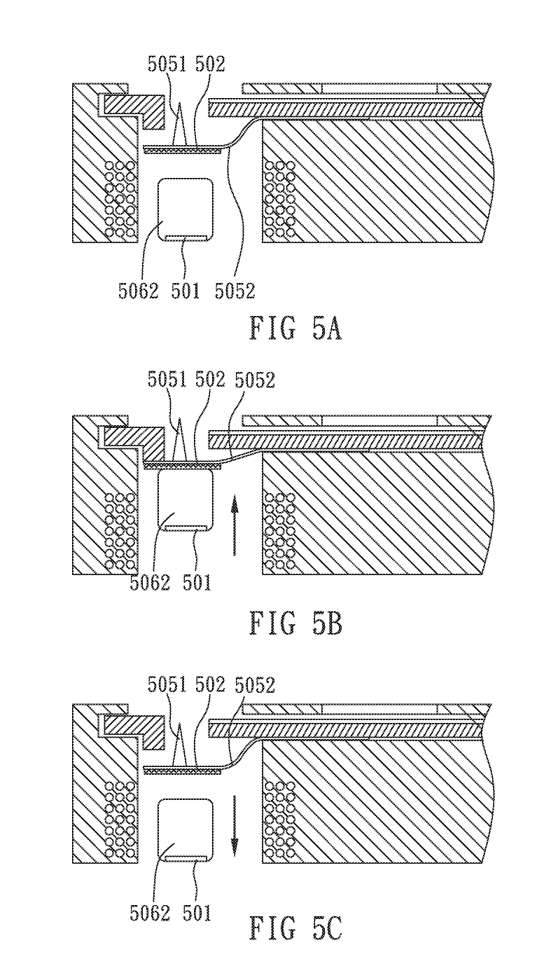

Please refer to FIG. 5A, FIG. 5B and FIG. 5C. FIG. 5A is the first schematic diagram of the driving unit of the blood component sampling equipment of example 5 of the present invention, FIG. 5B is the second schematic diagram of the driving unit of the blood component sampling equipment of example 5 of the present invention, and FIG. 5C is the third schematic diagram of the driving unit of the blood component sampling equipment of example 5 of the present invention.

The different implementation between the blood component sampling equipment of example 5 and the blood component sampling equipment of example 4 of the present invention is that the driving unit of the blood component sampling of example 4 is mechanical and the driving unit of the blood component sampling equipment of example 5 is electromagnetic.

Please refer to FIG. 5A. The moveable part 5062 is configured with a permanent magnet 501, and the supporting part 5052 is wound with a coil 502. A current is introduced into the coil 502 (not shown), and an electromagnetic field is induced (not shown) after introducing a current into the coil 502. Then, during the magnetic interaction, the moveable part 5062 is lifted as shown in FIG. 5B. At the same time, when the supporting part 505 is lifted by the push of the moveable part 5062 to the position as shown in FIG. 5B, the needle part 5051 penetrates into a patient's skin to collect the blood sample.

After the collection of the blood sample is finished, the current introduced into the coil 502 is shut down, and the moveable part 5062 is reverted back to its original position, as shown in FIG. 5A.

The above-mentioned embodiments are for the illustration only, the claims claimed in the present invention, which are not limited to the above embodiments.

What is claimed is:

1. A blood component detection unit, comprising:
a capsule body having a first surface;
a needle array including a plurality of needles, each of the needles having an opening and an inner hollow space, and each of the needles protrudes out of the first surface;
a detection chamber disposed inside the capsule body and connecting with the inner hollow space;
a sensing chip disposed inside the capsule body; and
a plurality of sensing elements disposed on the sensing chip.

2. The blood component detection unit as claimed in claim 1, wherein the blood component detection unit is in tablet shape.

3. The blood component detection unit as claimed in claim 1, wherein the blood component detection unit is a disposable blood component detection unit.

4. The blood component detection unit as claimed in claim 1, wherein the plurality of needles is made of biocompatible material.

5. The blood component detection unit as claimed in claim 1, wherein the capsule body is made of biodegradable material.

6. The blood component detection unit as claimed in claim 1, wherein the plurality of needles is coated with an anesthetic.

7. The blood component detection unit as claimed in claim 1, wherein the plurality of needles is manufactured by imprinting process.

8. The blood component detection unit as claimed in claim 1, wherein a mold for the imprinting process is manufactured by LIGA process.

9. A blood component detection device, comprising:
a strip having an upper surface; and
a plurality of blood component detection units as claimed in any one of claims 1 to 8, wherein the plurality of blood component detection units is disposed on the upper surface.

10. The blood component detection equipment as claimed in claim 9, wherein the plurality of blood component detection units is pasted on the upper surface.

11. The blood component detection equipment as claimed in claim 9, wherein the distance between two adjacent blood component detection units is the same.

12. A blood component sampling equipment, comprising:
a housing, having an opening;
a blood component detection equipment as claimed in any one of claims 9 to 11, which is disposed in the housing;
an elastic element disposed in the housing; and
at least one roller element transporting the blood component detection unit.

13. The blood component sampling equipment as claimed in claim 12, wherein the elastic element is a spring.

14. The blood component sampling equipment as claimed in claim 12, wherein the blood component sampling equipment further comprises a cap covering one end of the opening of the housing.

15. A blood component sampling equipment, comprising:
a housing;
a lid disposed on the housing;
a turntable component disposed inside the housing and having a central hole, wherein the turntable component rotates around the central hole as an axis;
a depth controlling component having a plurality of sub-sections, wherein each of the sub-sections has different height;
a plurality of blood component detention assemblies, each of the blood component detection assemblies comprises a needle section and a supporting section; and
a driving unit comprising a fixed part and a moveable part, wherein fixed part has a pushing part;
wherein the needle section is disposed on the pushing part.

16. The blood component sampling equipment as claimed in claim 15, wherein the turntable rotates in clockwise or counterclockwise direction.

17. A blood component sampling equipment, comprising:
a housing;
a lid disposed on the housing;
a turntable component disposed inside the housing and having a central hole, wherein the turntable component rotates around the central hole as an axis;
a plurality of blood component detention assemblies, each of the blood component detection assemblies comprising a needle section and a supporting section; and
a driving unit comprising a fixed part and a moveable part;
wherein the fixed part is wound with a coil, and the moveable part is configured with a permanent magnet.

18. The blood component sampling equipment as claimed in claim 17, wherein the turntable component rotates in clockwise or counterclockwise direction.
